(19) United States
(12) Patent Application Publication
(10) Pub. No.: US 2025/0069387 A1
(43) Pub. Date: Feb. 27, 2025

(54) REAL TIME AUTOMATED NERVE IDENTIFICATION SYSTEM

(71) Applicant: THE GEORGE WASHINGTON UNIVERSITY, Washington, DC (US)

(72) Inventors: Gary MILAM, Washington, DC (US); Chung Hyuk PARK, Falls Church, VA (US); Baijun XIE, Washington, DC (US)

(21) Appl. No.: 18/847,605

(22) PCT Filed: Mar. 24, 2023
(86) PCT No.: PCT/US2023/064945

§ 371 Date: Sep. 16, 2024

Related U.S. Application Data
(60) Provisional application No. 63/323,329, filed on Mar. 24, 2022.

(51) Int. Cl. G06V 10/94; A61B 5/00; G06V 10/147; G06V 10/26; G06V 10/60; G06V 10/80; G06V 10/82; G16H 30/40

(52) U.S. Cl. CPC G06V 10/955; A61B 5/4893; G06V 10/147; G06V 10/26; G06V 10/60; G06V 10/80; G06V 10/82; G16H 30/40; G06V 2201/03

(57) ABSTRACT

Precise nerve detection during surgery is crucial for safe operational outcomes, which largely depend on the surgeon's skills. Therefore, automatic real-time identification of anatomical structures is of great importance. However, image processing times must be kept as low as possible to be a viable solution for real-time visual identification. By using a general purpose graphics processing unit (GPGPU) implementation for birefringence mapping, the process can achieve rates of 43 frames per second (FPS); a 118×gain from a CPU implementation. Furthermore, by including a deep learning network, the complete framework can automatically detect nerves close to 12 FPS. By leveraging GPU for the processing task, the framework can run on compact devices with NVIDIA modules, such as Jetson Xavier AGX, while still achieving reasonably fast nerve identification and visualization.

### CROSS-REFERENCE TO RELATED APPLICATIONS

This application claims the benefit of priority of U.S. Application Ser. No. 63/323,329 filed on Mar. 24, 2022, the content of which is relied upon and incorporated herein by reference in its entirety.

### BACKGROUND

Neurogenic injury as a result of surgical mishap can present with significant morbidity and even mortality depending on the location of the injury. This may be attributable to the most common method for intraoperative nerve identification using direct visual observation or intraoperative electrical nerve stimulation. Relying only on human visual cues tends to magnify variations in clinicians' proficiencies, with outcomes that are strongly surgeon-dependent. This may result in a scenario where iatrogenic trauma is not uncommon. Reports show that 17% of the total number of nerve injuries (e.g., 126 of the 722 surgically treated cases) occur unexpectedly during surgical interventions (Kretschmer et al., 2001).

Injuries to motor neurons, in particular, can have critical impacts on the quality of life. For example, during head and neck surgery, facial nerve injury could result in facial paralysis that includes asymmetry of facial expressions, difficulties in eating or drinking, loss of blinking control, and drooping of the mouth on the affected side. Damages to the recurrent laryngeal nerve (RLN) during thyroidectomy, open neck, or cardiac surgery could induce paresis or palsy of the vocal cord with voice or swallowing dysfunction. Likewise, bilateral RLN injury could result in airway obstruction, resulting in the requirement for a tracheostomy. Pelvic nerve injury after a prostatectomy or rectal cancer surgery is associated with urinary and sexual dysfunctions. Finally, spinal cord surgery, as in the release of a tethered cord, can have dire consequences on bladder, bowel, or lower extremity motor function if the inadvertent neural injury occurs. The variety of postoperative nerve injuries described persisting even in the most experienced hands. Thus, there is an unmet clinical need for a noninvasive intraoperative nerve identification to increase situational awareness during operation.

The current surgical practice remains primarily passive and based on visual observation of anatomical reference points. Although a great deal of optical information is continually present in the operative field, human visual ability alone is spectrally-limited and polarization insensitive and therefore, abundant information is unobtainable to the unaided operator. Incorporating additional imaging sensors and screen display into the surgical workflow may seem relatively straightforward, similar to standard laparoscopy. However, in order to generate “super-human vision” capabilities and positively impact patient outcomes, the imaging sensors must produce actionable, active topographic guidance on critical anatomy along with situational awareness alerts, allowing surgeons to rapidly and intuitively localize unseen or hidden nerves.

As a standard of care, visual inspections have been performed by operating surgeons that rely on individual surgeon's experience and training. More recently, intraoperative neuromonitoring devices have been introduced and adopted for nerve localization (Cha et al., 2018). However, this technique requires intermittent electrical stimulations via an electrode probe to confirm the neuromuscular activity, which can interrupt surgical workflows. As an alternative, our group recently proposed and successfully demonstrated an optical imaging nerve identification using the Mueller polarimetric imaging (Ning et al., 2021), which calculates intrinsic birefringence patterns from fibrous nerve structures. In our previous study (Ning et al., 2021), the system showed promise but had a limitation of nerve-specific segmentation due to the presence of similar birefringence signals from other surrounding fibrous anatomies of tendons and collagenous muscle tissues. Therefore, we concluded that more advanced image processing with larger imaging data would further improve the specificity of the optical nerve identification.

In recent years, deep convolution neural networks (CNNs) have shown state-of-the-art results in many computer vision and medical imaging tasks. CNN has achieved remarkable results in the task of image classification. In addition to image classification, CNN has been designed to solve semantic segmentation tasks as well.

Despite the breakthrough of CNN in the computer vision and medical imaging domains, one drawback is that CNN structures usually require a massive amount of data to train. However, in the field of medical imaging, the acquisition process of images involves many layers of medical protocols and privacy/security issues, on top of which experts' annotations are also heavily required. These make the usage of CNN more expensive and time-consuming, even if the data becomes accessible.

In a recent study, the U-Net (Ronneberger et al., 2015) model alleviates this problem by using an encoder-decoder architecture. The encoder-decoder structure and the so-called “skip connection” enable the U-Net to get more prominent performance with a small amount of training data. Recent studies have also made improvements based on the U-Net and shown better performances in the semantic segmentation tasks. UNet++ consists of nested and dense skip connections to get more effective results (Zhou et al., 2018). Oktay et al. (2018) proposed the network with an attention gate (AG) module at the decoder part of the U-Net, which targets to learn the salient features for a specific task. The results showed better prediction performance on different datasets while preserving computational efficiency.

Current research efforts have also shown the effectiveness of employing U-Net to tackle the semantic segmentation task in medical imaging. Ultrasound nerve segmentation images dataset from the Kaggle competition has been widely used and trained by U-Net in many studies. The possibility of the adaptation of the generative adversarial network (GAN) has also been discussed. Moreover, U-Net has been used to identify more complicated nerve structures from ultrasound images that contain the musculocutaneous, median, ulnar, and radial nerves, as well as the blood vessels. Other studies have also deployed the U-Net model to identify other nerve structures, such as the proposed corneal nerve segmentation network, for sub-basal corneal nerve segmentation and evaluation.

### SUMMARY

The present disclosure is primarily motivated by the limitations of current nerve identification techniques, which can be time-consuming, invasive, and error-prone (Henry et al., 2017). One current surgical approach is electrical nerve stimulation, the so-called intraoperative effector muscle monitoring (IONM), which, while effective, is invasive and can create distractions during surgical procedures. Additionally, it only works with motor neurons: sensory branches can be inherently missed during stimulation. Alternatively, recent fluorescent markers (including clinically approved methylene blue, indocyanine green, ICG) can be used to highlight relevant nerve structures. However, at present, the use of these markers is still experimental, and concerns remain regarding toxicity. Thus, the most common method for nerve identification remains direct visual observation by operating surgeons or IONM. The ideal solution would be a noninvasive vision assistant tool that can simultaneously address the limitations of current technologies and human factors.

This disclosure introduces an improved U-Net architecture for nerve identification, the Dual Cross-Modal Transformer Fusion U-Net (DXM-TransFuse U-Net), which has two different modality input paths fused by a transformer to have an integrated decoder output path. The novelty of our CNN is leveraging the characteristics of the Transformer to model the interactions and relationships between different modalities of images. The disclosure also demonstrates the eminence of using the birefringence map for the task of image segmentation. This is the first use of birefringence images for the task of biomedical image segmentation with deep learning. The results of the single modality experiments confirm that the birefringence map is a favorable choice for the task of biomedical image segmentation. For the comparison study, we compare our method with the existing state-of-the-art fusion techniques and show the superiority of our model based on detection and segmentation metrics, model complexity, and inference time. These significant findings suggest that our proposed multimodal imaging system can identify nerves in real-time, which is ideal for surgical procedures.

Intraoperative identification of clinical structures such as nerves and vessels is paramount to achieving successful surgical outcomes and decreasing potential post-operative complications. For example, iatrogenic nerve injury can be the result of direct surgical trauma or mechanical stress. Surgeons greatly rely on their memory and extensive anatomic knowledge to identify nerve structures intraoperatively, which can be challenging given multiple anatomic variations within the general population. In addition, nerves can be damaged due to not being clearly exposed in the operative field or being mistaken for a tendon or vessels. In large-scale studies, 25% of sciatic nerve lesions that required treatment were iatrogenic, as were 60% of femoral nerve lesions and 94% of accessory nerve lesions [1].

Intraoperative nerve injury can lead to loss of function, sensation, muscular atrophy, and/or chronic neuropathy, considerably impairing patient quality of life [2]. For instance, thyroidectomy is one of the most common surgeries performed, particularly in countries where iodine deficiency is a common condition [3]. The most serious postoperative complication of thyroidectomy is recurrent laryngeal nerve (RLN) paralysis that leads to vocal cord palsy with subsequent temporal or permanent voice change [4]. In reviewing multiple studies on unilateral RLN injury, surgery is cited as the most common cause, with most studies putting it as the cause of 30 to 40% of all RLN injuries [5].

Currently, nerve-sparing techniques consist of the identification of anatomic landmarks and electrophysiologic monitoring modalities, including electromyography (EMG), somatosensory evoked potentials (SSEPs), brainstem auditory evoked potentials (BAEPs), motor evoked potentials (MEPs), among others [6]. These neuromonitoring techniques are commonly used in the operating room to improve surgical decision-making and possibly reduce complications. However, certain limitations exist for each technique, including intraoperative placements of neural probes [2], signal averaging results in time delay, and a high rate of false positives [7].

Dye-based nerve highlights [8] or using Raman spectroscopy [9] are not suitable for surgical procedures with humans, and optic-based nerve localization methods during surgery have been investigated but the processing time has not been real-time. The deep learning approaches were also explored for detecting nerves, such as third molars and mandibular nerve [11], and the U-Net architecture, which is a convolutional neural network (CNN) for medical imaging, was found to achieve encouraging results. However, the lack of contrast of the orthopantomogram imaging and the shortage of annotated data can degrade the performance of the segmentation. Other variants of CNN structure have also been applied for recognizing nerve tissues with a noninvasive imaging technique [13]; nonetheless, the sensitivity and specificity of classifying the nerve tissues were unsatisfactory. The inadequacies of existing nerve identification methods, which can be time-consuming, intrusive, and fallible [14], are the main driving force for our proposed work. Therefore, the development of a nerve detection technique that is non-invasive, cost-efficient, accurate, and with real time feedback capabilities is important to prevent intraoperative neurologic complications.

In one embodiment, a nerve detection or identification system takes multi-modal camera sensor inputs (RGB color image+polarimetric image) in camera stream, generates birefringence maps of the polarimetric image and also transforms the color image to match the view area, and then feed into a deep learning architecture that takes these multimodal input image streams to process over dual cross-modality fusion transformer network with U-Net. Our system also helps the user to provide easier ground truth masking, and more ground truth data can be used to further enhance the system performance. The image processing and deep learning framework is also integrated into the general-purpose graphic processor unit (GPGPU) architecture to enable real-time detection and visualization of nerve tissues from the camera system, enabling real-time surgical scene visualization with nerves marked.

### BRIEF DESCRIPTION OF THE DRAWINGS

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1 shows the overall system for nerve detection with GPU enabled;

FIG. 2 is a birefringence mapping calculations from the prior art;

FIG. 3 is an execution flow comparison between CPU (FIG. 3(a)) and GPGPU (FIG. 3(b));

FIG. 4 shows samples of the generated images and masks;

FIG. 5 is a block diagram of the Dual U-Net with the cross-modal Transformer block module;

FIG. 6 is a cross-modal Transformer block;

FIG. 7 is an overview of the real time nerve detection process with GPGPU and DXM-Fusion network [15];

FIG. 8 shows predicted mask comparisons of single modality (Upper two rows: birefringence images, lower two rows: RGB images);

FIG. 9 shows predicted mask comparisons between single modality and the proposed multi-modality networks;

FIG. 10 shows predicted mask comparisons among different multi-modality networks;

FIG. 11 shows Layer Grad-Cam attributions for each encoder path before transformer block and post transform-fusion after bottleneck (cyan color pixels represent ground truth mask of the nerve);

FIG. 12 shows estimated frames per second for different data types;

FIG. 13 shows image outputs based on double and float precision;

FIG. 14 shows a comparison of total execution times with Deep Learning Networks on Xavier AGX Developer Kit; and

FIG. 15 is an estimated FPS for complete flow of mask predictions.

The figures show illustrative embodiment(s) of the present disclosure. Other embodiments can have components of different scale. Like numbers used in the figures may be used to refer to like components. However, the use of a number to refer to a component or step in a given figure has a same structure or function when used in another figure labeled with the same number, except as otherwise noted.

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

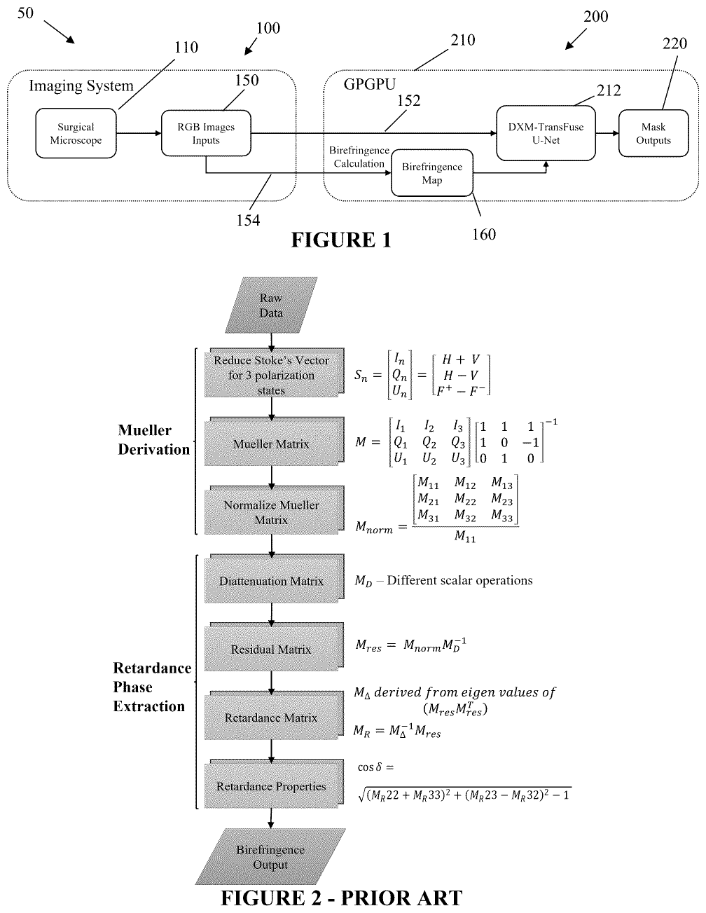

Turning to the drawings, FIG. 1 illustrates a non-limiting embodiment of the disclosure comprising an image identification system 50 that performs detection, visualization and/or analysis of body features, and especially nerves. The system 50 comprises a multimodal imaging system 100 and a medical image segmentation module 200. The multimodal imaging system 100 comprises an imaging apparatus 110, and the segmentation module 200 comprises a general-purpose graphics processing unit (GPGPU) 210. The imaging apparatus 110 can include, for example, a surgical microscope 110 and an imaging device such as a camera or image sensor. The imaging apparatus 100 collects images 150 and provides them to the GPGPU 210. In some embodiments, the microscope 110 is a dual camera system and outputs RGB image data 152 and raw polarized data 154. The GPGPU 210 processes the raw polarized data 154 to compute a birefringence maps 160 (i.e., Jet image data). The raw RGB data 152 is provided to the DXM-TransFuse U-Net, which fuses the raw RGB data 152 with the birefringence map 160.

Birefringence calculation is designed to get optical properties of a material in terms of a refractive index with regards to the polarization and propagation direction of light, and the birefringence map is a 2D representation of the birefringence indexes over the imaging area. The birefringence map is used as another modality of input which is different from RGB image modality. The DXM-Transfuse U-Net combines information from both modalities (i.e., the raw RGB image data 152 and the Jet image data 160) to make more accurate predictions. The RGB image data can be specially lighted, for example, to create the birefringence map 160.

The medical image segmentation module 200 comprises a GPU embedded device having a GPGPU 210. The medical image segmentation module 200 is in wired or wireless communication with the multimodal imaging system 100. In some embodiments, due to the heat of the system, the GPGPU 200 is placed externally to the imaging system 100 with a cooling system; though in some embodiments the GPGPU 200 can be local and/or integrated with the imaging system 100. The GPU embedded device has a neural network design. The neural network design is separate from the GPU device. The GPGPU 210 includes the DXM-TransFuse U-Net module 212. The DXM-TransFuse U-Net 212 accepts the medical images 152, 154 and outputs the mask images 220, which indicate the nerves. The GPGPU 210 receives the images 152, 154 and the birefringence maps 160 from the GPGPU 120, and predicts segmentation outputs, e.g., a mask output 220, based on the RGB image data 152 and the birefringence map (Jet data) 160. The mask output 220 can identify the position and shape of the nerves based on the input images. It can be used for assisting to identify a surgeon during surgery, and out system can process that in real-time. The mask outputs 220 can indicate the position and the shape of the nerve.

The image detection, visualization and analysis system 50 operates in real-time to achieve nerve detection and achieves a 118+times boosted birefringence map generation process using general-purpose graphics processing unit (GPGPU) and a deep learning-based nerve tissue identification network (DXM-TransFuse U-Net [15]). The neural network design is a machine learning method that can make prediction (i.e., maximizes the nerve detection (classification) output; that is, the percentage that the image portion is a nerve to predict where the nerve exists in an image) based on the input data. In our case, the inputs are the images 152, 154 from the surgical camera 110 and the outputs are the mask images 220 indicating the nerves. The acceleration is achieved through the parallel computing of GPU.

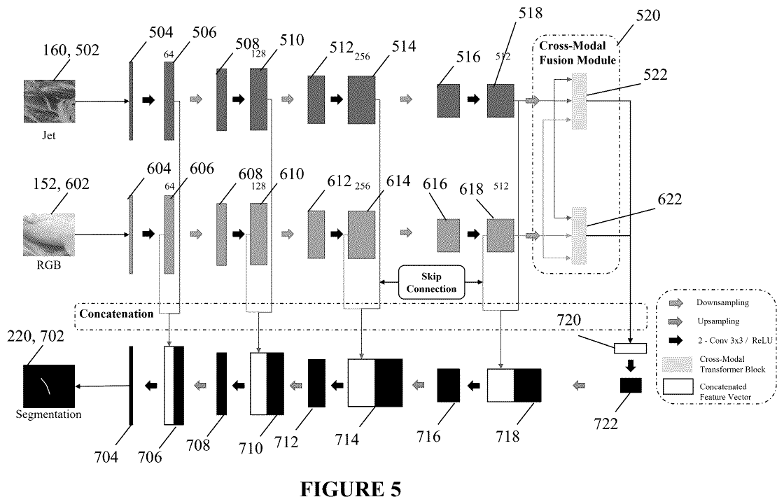

Referring to FIGS. 1, 5, the network is designed to leverage birefringence 160 and RGB images 152 on which the feature maps of each modality are extracted independently, and the information is fused at the bottleneck with a Transformer block for cross-modal interactions. The bottleneck is the term used in the U-Net 212. It is a way to force the model to learn a compression of the input data. The extracted outputs 518, 618 (FIG. 5) are the compressed data. The idea is that this compressed view should only contain the ‘useful’ information to be able to reconstruct the segmentation map 220, 702. We fused two different modalities of that ‘compressed’ data and modeled the interaction through 520, as handled by the GPGPU 210. The inputs were extracted independently as shown at 502-518 and 602-618, and they were leveraged through the 520. Blocks 504-518, 604-618 encode the image data, for example to downscale the image data (e.g., by eliminating immaterial information and retaining the important features that might represent key characteristics (nerve features) of the original image data 150, 160) and provide the compressed image data 518, 618. Steps 718-704 extrapolate (decode) the image data, and the final segmented image mask outputs 220 are two images.

The Transformer blocks 522, 622 receive the compressed image data 518, 618 and fuse the compressed Jet image data 518 and the compressed RGB image data 618 to provide a modified (dual links) concatenation bridge image 720 in a typical U-Net structure between encoding and decoding (or compression and extrapolation) layers. In our dual cross-modal transformer-based fusion (DXM-TransFuse) network, the image 720 handles information from both input modalities. The transformers 522, 622 models the fusion and leverage the ‘useful’ information from both modalities.

In addition, the system 50 uses raw polarized data to achieve real-time nerve detection and visualization using polarimetric imaging system [2]. As well, the imaging system 110 captures the RGB image which is used with the resultant birefringence image as inputs to the DXM-Transfuse U-Net providing the final nerve segmentation mask.

More recent studies are exploring the possibility of using U-Net based structure to combine different image modalities for training (Dolz et al., 2018a,b; Kumar et al., 2020). A multi-path architecture is employed that can extract and combine the unique features from different modalities (Dolz et al., 2018a,b). Kumar et al. (2020) have presented a new CNN that aims to fuse complementary information in multi-modality images for medical image segmentation. That study introduces the co-learning component that allows the model to learn the fusion of different modalities and the importance of a specific modality by placing weight accordingly to the feature maps of each modality.

Since salient parts in medical imaging can vary greatly, Dolz et al. (2018a,b) proposed an extended inception module that would be able to learn from different receptive fields. Using convolutions of multiple kernel sizes at the same level allows for capturing both local and general information; furthermore, two additional dilated convolution blocks with different dilated rates were leveraged to help increase the captured global context. Although the architectures proposed by Dolz et al. (2018a,b) leverage extended inception modules and dense connectivity between multiple paths of the encoder to show improvements over other fusion techniques, the increase in model size and the potential delay in the inference time that comes along with those models are still existing limitations.

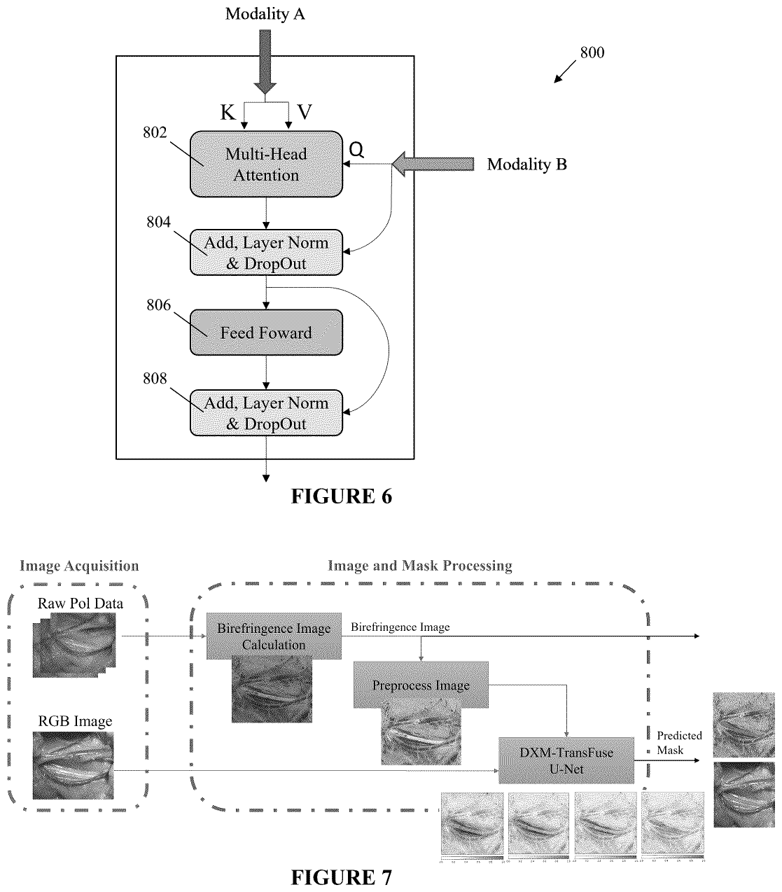

Therefore, the present disclosure leverages attention mechanisms to focus on the region of interest and learn the importance of the higher-level features of each modality to increase the model's prediction capabilities over other single U-Net variants while maintaining the size and inference time at an acceptable range. The attention mechanism is a method to address the bottleneck problem in machine learning, which is realized at the cross-modal fusion module 520. The attention mechanism is incorporated in the transformer blocks 522, 622. A recent study shows that the attention mechanism purely based on multi-head attention (Vaswani et al., 2017) has significant advantages, which has also been applied to the task of medical image segmentation (Petit et al., 2021). A study proposed by Petit et al. (2021) also added the multi-head attention module at each skip connection and demonstrated better performance than the previous study of attention gates mechanism (Oktay et al., 2018). However, existing studies have only focused on applying Transformer for U-Net on the unimodal dataset. Few studies have used the Transformer for multi-modal fusion in the task of medical image segmentation. The usage of Transformer for cross-modal interaction has been proposed and applied mainly in the study of natural language processing (Tsai et al., 2019). FIG. 6 is a visualization of the transformer blocks 522, 622.

Thus, the system 50 improves the performance of the intra-operative nerve identification system by using multi-modal medical images with deep neural networks. Here, multi-modal refers to using both RGB and birefringence as the inputs for the neural network. The ‘deep’ is important because conventional segmentation methods cannot gain the performance as deep neural network can achieve. This system 50 presents an improved Dual Cross-Modal Transformer Fusion U-net (DXM-TransFuse U-net), which uses a multi-path architecture that learns to fuse different image modalities via cross-modal interactions, where each modality of the image is treated as an independent signal. Furthermore, a Transformer module (e.g., Tsai et al. (2019)) is implemented at the bottleneck for the fusion of different modalities. One significance of the system 50 lies in designing the enhanced U-Net architecture and analyzing and comparing the performance of the different combinations of image modalities with actual nerve tissue data collected during intraoperative processes. In addition, we conduct experiments to demonstrate that the system 50 is more efficient compared with other baseline models.

### Birefringence Images

Birefringence images 150 are derived from a polarimetric camera using Mueller matrix decomposition, described in our earlier work (Cha et al., 2018). The output provided by the multi-modal optical imaging system can include RGB and birefringence outputs (Jet and Grayscale), overall consisting of three types of images 150: RGB, Jet, and Grayscale. As shown in FIG. 4, the first three columns display the three image types with different modalities, with the last column displaying the ground-truth mask annotated by the expert. As the two birefringence image types are of the same source, we would only be using a kind for our models, with the Jet representation being chosen because it consists of 3 channels and carries more information that would be significant during training.

To improve the processing times of the birefringence mapping, the calculations were ported from a CPU implementation to a GPGPU. Since calculations of a birefringence pixel are independent of other pixels, the task can be parallelized, and GPGPU implementations are perfectly suited for it. Furthermore, the Jet color mapping was performed directly during the birefringence calculation of the pixel, as opposed to post birefringence image processing. These changes increase the processing speed by 140×, going from 2430 ms (ms) for a CPU implementation to 19.47 ms for the GPGPU implementation.

Ground-truth masks were annotated by two experienced surgeons after the confirmation of the Neuromonitoring device (Nerveana Nerve Locator and Monitor, Neuro Vision Medical Products, Ventura, CA). The last column of FIG. 4 shows the samples of expert annotated masks in this study. A user-oriented masking software was used. In addition, the marker-based watershed algorithm provided by the OpenCV library was implemented on the software to provide the annotators with the option to use the watershed to assist in mask creation.

The used dataset is curated to ensure images from all modalities are in line with each other, as this would be essential for the multi-modal network approach. The entire dataset consists of 188 images of each modality: birefringence (Jet) and RGB images.

The dataset is then divided into five groups for establishing the 5-fold cross-validation procedure, which is desired in the absence of a large dataset. For preprocessing, images are resized to 256×256, with standardization and normalization techniques applied. Normalization is performed using the means and standard deviations of the channels of their respective image types.

Positional and color augmentation techniques are performed on each image of the training set and are added to the set. Training images are vertically flipped with the brightness randomly modified, horizontally flipped with contrast randomly adjusted, rotated at a randomly selected degree of multiples of 90 degrees, and applied with Gaussian noise; only positional augmentations are performed on the respective masks.

### Birefringence Mapping

Birefringence mapping 160 is calculated at the GPGPU 210 (FIG. 1) from raw polymetric data by deriving the 3×3 Mueller matrix and extracting the phase retardance as described in [16]. The flow chart shown in FIG. 2 describes the steps included for the Mueller derivation and the extraction of the phase retardance properties for the birefringence pixel output for birefringence mapping. Since the calculations of each birefringence pixel output are performed independently, it makes it a prime candidate for parallel computation. On a CPU implementation, instead of performing each calculation for each pixel in a sequential fashion, each calculation step can be done leveraging an N×3×3 matrix, where N represents the number of output birefringence pixels, improving the efficiency of the overall process. However, the number of threads available in a GPU are much greater than that available in a CPU, and since all calculations for an individual pixel are the same and are independent of each other, the calculation tasks can be done through a GPGPU implementation.

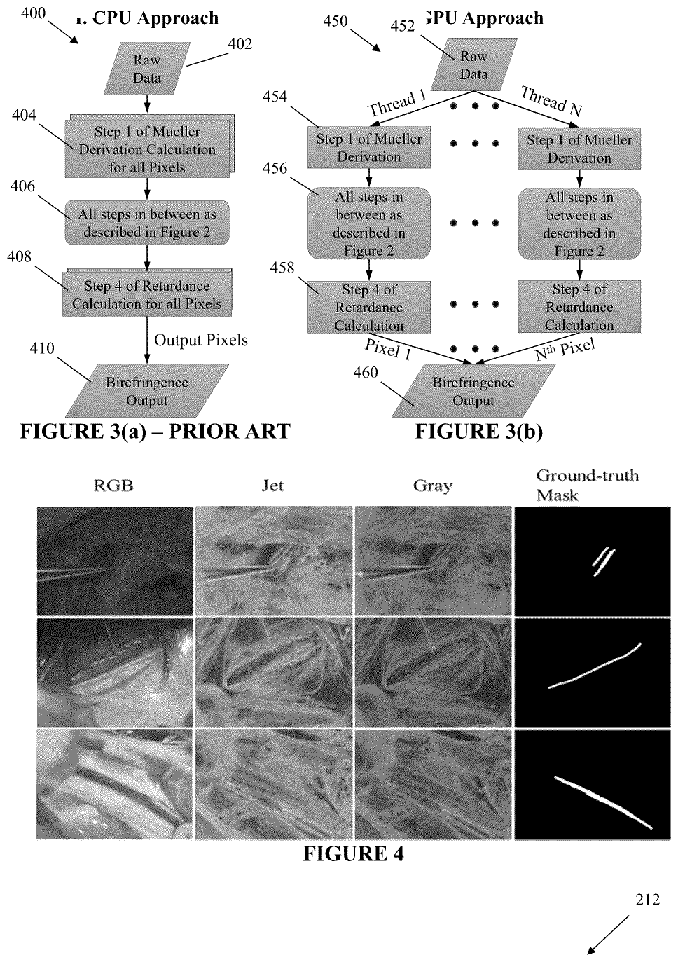

FIG. 3 is a flow comparison between a CPU flow 400 (FIG. 3(a)), and a GPGPU flow 450 (FIG. 3(b)). The calculations begin from Reduce Stoke's vector matrices which are of 3×3 size. The matrix operations performed on these matrices include multiplication, transposition, inversion, and calculating the eigenvalues. Due to the combination of operations and dealing with many (N) small matrices, it would be best suited if the calculations are approached in a single-threaded fashion while parallelizing the task. The GPGPU approach can be leveraged to perform the birefringence calculations for each output pixel in parallel, as opposed to the CPU implementation, where each calculation is performed for all pixels at each step in the sequence. FIG. 3 provides a comparison between the two approaches, namely a Computer Processing Unit (CPU) (FIG. 3(a)), and a GPGPU (FIG. 3(b)). Birefringence images are derived from a polarimetric camera using Mueller matrix decomposition, described in our earlier work (Cha et al., 2018). The CPU approach (FIG. 3(a)) conducts calculations 400-410 serially; whereas the GPGPU (FIG. 3(b)) conducts calculations 450-460 in parallel. The CPU can do multi-threading but not parallel processing. Multi-CPU can be another approach, but the CPU process of accessing memory externally is not as fast as GPGPU process. The parallel computing by the GPU (FIG. 3(b)) takes the birefringence mapping process that is resource intensive and too slow for real-time usage and parallelizing the pixel computations into individual pixel tasks that would make it viable for real-time processing.

The multi-modal optical imaging system provides RGB and birefringence outputs (Jet and Grayscale), overall consisting of three types of images: RGB, Jet, and Grayscale. As shown in FIG. 4, the first three columns display the three image types with different modalities, with the last column displaying the ground-truth mask annotated by the expert. As the two birefringence image types are of the same source, we would only be using a kind for our models, with the Jet representation being chosen because it consists of 3 channels and carries more information that would be significant during training.

Ground-truth masks were annotated after the confirmation of the Neuromonitoring device (Nerveana Nerve Locator and Monitor, NeuroVision Medical Products, Ventura, CA). The last column of FIG. 4 shows the samples of expert annotated masks in this study. A user-oriented masking software was used. In addition, the marker-based watershed algorithm provided by the OpenCV library was implemented on the software to provide the annotators with the option to use the watershed to assist in mask creation.

The used dataset is curated to ensure images from all modalities are in line with each other, as this would be essential for the multi-modal network approach. The entire dataset consists of 188 images of each modality: birefringence (Jet) and RGB images.

### Deep Learning Application

Current research has shown that deep learning networks can be employed to effectively identify nerve structures using coherent anti-Stokes Raman scattering (CARS) endoscopic images to potentially assist during intraoperative surgery. However, as pointed out by the study, the acquisition time for CARS endoscopic imaging can be in the order of minutes. Another study had leveraged Hyperspectral imaging (HSI) as inputs to a deep learning network, but the acquisition time for HSI is around 10-15 seconds. Therefore, there is a need for images that can be quickly processed and used as inputs to a neural network to identify nerve structures in real-time.

### Deep Neural Network Architecture

To demonstrate the effectiveness of the proposed multi-modal deep neural network, we first compare the proposed architecture to the single-modality network. For single-modality, U-Net (Ronneberger et al., 2015) and Attention U-Net (Oktay et al., 2018) are considered the baseline of the study. Original U-Net contains an encoder and a decoder, which is developed for biomedical image segmentation. The encoder part is called the contraction path, which is consecutive stacking with convolutions followed by max-pooling layers for down-sampling. The decoder part is the expansion path which is up-sampling by up-convolution. The U-Net uses a skip connection to address the problem of losing information during the up-sampling, which integrates the information from the contraction path.

Attention U-Net differs from U-Net by adding soft attention gates at the skip connections (Oktay et al., 2018). The attention mechanism enables the network to focus on the region of interest and reduces redundant features. Following the derivation of Oktay et al. (2018), xl is the output feature map from the previous layer. The gating signal g is collected from a coarser scale that provides contextual information for the focus spatial regions. The attention coefficient, a, is used to identify salient features and suppress redundant features to reserve the activations relevant to the specific task. The attention coefficient is formulated as follows:

$$α = {σ}_{2} ( {ψ}^{⊤} ( {σ}_{1} ( {W}_{x}^{⊤} {x}_{l} + {W}_{g}^{⊤} g + {b}_{g} ) ) + {b}_{ψ} ) \qquad ( 1 )$$

where σ1 is the ReLU function, σ2 is the Sigmoid function, Wx, Wg, and ψ are linear transformations, and bg and bψ are the bias terms.

The output of AGs xout is the element-wise multiplication of input feature map xl and the attention coefficient a:

$${x}_{out} = {x}_{l} \cdot α \qquad ( 2 )$$

The cross-attention module introduced in Petit et al. (2021) is investigated as another attention mechanism for the skip connections. Similar to Attention U-net, it is used to put focus on the regions-of-interests (ROIs) while suppressing irrelevant information. The module uses a multi-head attention module that uses as input the higher-level feature maps from the previous layer and the skip connection information. A sigmoid activation is then applied to the computed values, which is then followed by an element-wise multiplication of it with the skip connection.

### Multi-Modal Deep Neural Network Architecture

Two fusion approaches are investigated for the multi-modal baseline comparison. One is a late-concatenation fusion approach at the bottleneck of the U-Net, and the other is adapting the co-learn module proposed by Kumar et al. (2020) at the bottleneck. The late-fusion approach was chosen over early-fusion as it is seen to outperform early fusion in previous studies, such as this study by Dolz et al. (2018b). Furthermore, the co-learn module is implemented at the bottleneck as opposed to each layer to allow fair comparison across all three fusion approaches and maintain the model at a reasonable size.

FIG. 5 is a block diagram of the Dual U-Net with the cross-modal Transformer block module. FIG. 5 shows the network architecture in accordance with an illustrative, non-limiting embodiment of the disclosure, in which two different modalities of the image are fused to learn the model. Two modalities, 502 and 602 are combined to get the final output 702. The processes of 504-518 and 604-618 are the same as the general U-net 212, which is a sequence of convolutional operations, and 722-704 is the sequence of retrieving mask output. At module 520, different modalities of inputs are fused to achieve higher performance compared with single modality. The parallel encoder structure allows the model to extract and combine the visual features of each independent modality. The base encoder is similar to the U-Net (Ronneberger et al., 2015), which aims to generate and down-sample the feature map. Then we adapt the Transformer with the multi-head attention module proposed by Vaswani et al. (2017) at the bottleneck. The multi-head attention module has been used for creating the self-attention feature map for image segmentation (Petit et al., 2021) but for a single modality. This study extends the idea and introduces the Transformer block for fusing different modalities of inputs via a cross-modal Transformer block.

FIG. 6 is a cross-modal Transformer block. As shown, the expression of the attention output is given as follows:

$$Attention ( Q , K , V ) = softmax ( \frac{{QK}^{⊤}}{\sqrt{{d}_{k}}} ) V , \qquad ( 3 )$$

where Q ∈

$${R}^{d \times {d}_{B}}$$

is the queries from the modality A, K ∈

$${R}^{d \times {d}_{A}}$$

is the set of keys, and V ∈

$${R}^{d \times {d}_{A}}$$

is the set of values from the modality B. The expression in the softmax function in Equation (3) measures the similarity of Q with respect to K. Then, the attention function 802 measures the information from modality B and modality A by calculating the products of the softmax function and V.

Following the setting from the previous study (Vaswani et al., 2017), we add a residual connection 804 and combine it with the layer normalization and dropout operations 804. Furthermore, a feed-forward network 806 with two hidden layers and another residual connection 808 with the layer normalization and dropout are added for the Transformer block.

### Multi-Task Learning

A Loss function combined with an equal weight of the Binary Cross-Entropy (BCE) and Edge-Loss functions is used to optimize the model in this study.

The BCE is implemented with weight placed on positive examples to help with the imbalanced nature of the dataset. The BCE loss function with a positive weight is formulated as:

$${ℓ}_{( Y , P )} = - ( 1 / N ) {\sum }_{n = 1}^{N} {w}_{p} {y}_{n} log ( {p}_{n} ) + ( 1 - {y}_{n} ) log ( 1 - {p}_{n} ) , \qquad ( 4 )$$

where yₙ Y denotes the target labels and pₙ P are the predicted probabilities for the nth pixel in the batch, with N being the number of pixels in the batch. The Sigmoid activation function is used for the predicted probabilities. The weight placed on positive examples is denoted as wₚ in the loss function and calculated as follows:

$${w}_{p} = \frac{TotalBackgroundPixelsOfSet}{TotalForegroundPixelsOfSet} . \qquad ( 5 )$$

Positive weight is calculated on each individual training run and based on the ground truth masks of the current run's training set.

Following the definition, the Edge-Loss function is given by:

$${ℓ}_{edge} = \frac{1}{C} \frac{1}{K} \frac{1}{N} {\sum }_{c = 1}^{C} {\sum }_{k = 1}^{K} {\sum }_{π = 1}^{N} {‖ {{E}_{k} ( y )}_{π}^{c} - {{E}_{k} ( p )}_{π}^{c} ‖}_{2} , \qquad ( 6 )$$

where C is the classes of segmentation, K is the number of Sobel kernels, N is the pixel number of the image, y is the label map and p is the prediction map. The final loss function to optimize the model is the sum of the BCE and Edge-Loss functions:

$${ℓ}_{total} = {ℓ}_{( Y , P )} + {ℓ}_{edge} . \qquad ( 7 )$$

### Network and Training Implementation

All single modality networks are made up of 4 paired encoder-decoder layers and the bottleneck. The ReLU activation function was used after each convolution except at the attention gate, where the sigmoid activation function is used to obtain the attention coefficient, according to formula 1. Additionally, the sigmoid activation function is used in the multi-head cross-attention module, as described by Petit et al. (2021).

Similar to single-modality networks, the multi-modality networks have four paired base layers, using the ReLU activation function after each convolution. However, the Co-Learn U-Net includes the co-learn module right before the bottleneck on the encoder path. This module contains a ReLu activation where the resultant is the co-learned fusion map. In addition, the DXM-TransFuse U-Net has an added layer on the encoder path to perform the cross-modal interaction by the transformer blocks, as seen in 4. In the feed-forward layer of the transformer block, a ReLu activation function is used between the two linear transformations as described in Vaswani et al. (2017).

Additionally, the transposed convolutions on the decoder path had been substituted with a bicubic up-sampling followed by a convolution operation. Although the bilinear or bicubic up-sampling are more expensive than transposed convolutions, they have shown to suffer less from artifacts. The bicubic method is chosen over the bilinear because sixteen nearest neighbors are used on the former instead of only four nearest neighbors on the latter. In addition, leveraging more information during the up-sampling will produce better segmentation results.

The stochastic gradient descent (SGD) optimizer with a multi-step decayed learning rate is employed to train the network architectures. An initial learning rate of 0.03 decayed by ⅓ at 0.25 and 0.75 of the total number of epochs (250) is implemented.

Eight images of each modality are used for each mini-batch. Code implementation was performed in PyTorch, and experiments ran on four Nvidia Tesla V100 SXM2 16 GB GPUs.

### Evaluation

To evaluate the models in this study, two types of metrics are used: one to evaluate the segmentation quality and the other to evaluate the detection of a nerve. A threshold of 0.5 is used for the predicted segmentation probabilities.

For evaluating segmentation quality, the two main metrics used are the Dice coefficient and the F2 score, which are given as follows:

$$DiceCoefficient = \frac{TP}{TP + \frac{1}{2} ( FP + FN )} , \qquad ( 8 )$$

$$F 2 Score = \frac{TP}{TP + \frac{1}{5} FP + \frac{4}{5} FN} . \qquad ( 9 )$$

where TP is true positive, FP false positive, FN false negative, and TN true negative.

Additionally, the Dice coefficient values are used to help with the metrics used for the evaluation of nerve detection: accuracy, sensitivity/recall, specificity, precision, and balanced accuracy, which are given as follows:

$$Accuracy = \frac{ImageswithDice > Dicethreshold}{TotalImages} , \qquad ( 10 )$$

$$Sensitivity / RecallRecall = \frac{TP}{TP + FN} , \qquad ( 11 )$$

$$Specificity = \frac{TN}{TN + FP} , \qquad ( 12 )$$

$$Precision = \frac{TP}{TP + FP} , \qquad ( 13 )$$

$$BalancedAccuracy = \frac{Sensitivity + Specificity}{2} . \qquad ( 14 )$$

To help with the classification metrics, the binary labels are set as part of the dataset to determine if an image contains nerve tissues or not. If the Dice value of the predicted mask is greater than the Dice threshold, this is considered a true positive if it is labeled as containing nerve tissues; otherwise, it is regarded as a true negative. If the Dice coefficient is below the threshold, it is considered false negative if it is labeled as containing nerve tissues; otherwise, it is considered false positive.

Results are based on the 5-fold cross-validation, where one-fifth is the hold-out set used for validation, with the remaining used for training. For the experimental comparisons, we performed the paired test with the p-value of 0.5.

In addition, Grad-Cam attributions at different layers were investigated to help with interpreting the model. The Captum library was used for this purpose.

The deep learning network, DXM-TransFuse U-Net shown above and [15], leverages a Jet representation of a birefringence image and its respective RGB image to identify nerve tissues automatically. The study had demonstrated that using the birefringence image alone had an improvement of the F2 score of at least 14% over its RGB counterpart. By fusing the two modalities through a transformer block, further improvements to the segmentation task were achieved.

The network architecture is shown in FIG. 5, in which two different modalities of the image are fused to learn the model. The parallel encoder structure allows the model to extract and combine the visual features of each independent modality. The base encoder is similar to the U-Net (Ronneberger et al., 2015), which aims to generate and down-sample the feature map.

Then we adapt the Transformer with the multi-head attention module proposed by Vaswani et al. (2017) at the bottleneck. The multi-head attention module has been used for creating the self-attention feature map for image segmentation (Petit et al., 2021) but for a single modality. This study extends the idea and introduces the Transformer block for fusing different modalities of inputs via a cross-modal Transformer block.

FIG. 7 is an overview of the real time nerve detection process with GPGPU and DXM-Fusion network, and illustrates the overall process for the automated nerve identification. After the raw polarized data has been acquired, the birefringence mapping is calculated. The output will then be normalized, and a Jet colormap is applied to it. The image is then resized to 256×256 and standardized. The resultant output is passed to the DXM-TransFuse U-Net with its respective RGB image, also resized to 256×256 and standardized, for the final mask prediction.

### Implementation

The GPGPU birefringence mapping code was implemented using with all calculations performed through a custom CUDA kernel. The code portion relevant to finding the eigenvalues of a matrix is based on the Jacobi Eigen library For the purposes of this study, the grid dimensions were set as (16,16,1) and the block dimensions as (128,153). Tests were done on a Windows 10 Intel® Core™ i9-9900K CPU @ 3.60 GHz with NVIDIA Geforce RTX 2080 Ti GPU. The CUDA version used on this device is 11.1.

Further tests were done on a Jetson AGX Xavier Developer Kit to determine the feasibility of performing birefringence mapping and deep learning inferences within acceptable times on a compact device. The CUDA version on that device was 10.2.

The raw polarimetric data provided for assessing the speed and reliability of the GPGPU calculations was acquired from a proprietary dual-RGB/polarimetric imaging system (unpublished) similar to the work [2]. The original code used as the base CPU implementation comes from the same.

The raw data consists of a matrix of size 3×2048×2448, from which the birefringence output results in a matrix of size 1024×1224. It is worth noting that the raw data matrix can be set to flip the first and last dimension during the image acquisition to end up with a 2048×2448×3 matrix. This was done for this study to leverage the CUDA vector built in data types (double3 or float3) for convenience purposes when calculating the reduced Stoke's vectors.

The deep learning network was implemented in PyTorch. All inference tests for the network were done on GPU; additionally, the tests were done using Float 32 and Float 16 data types for comparison purposes.

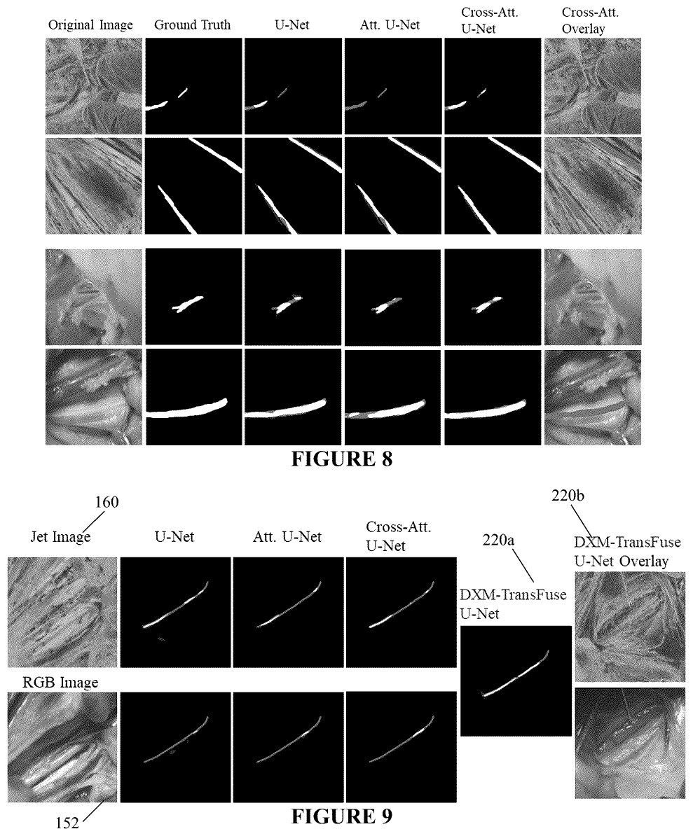

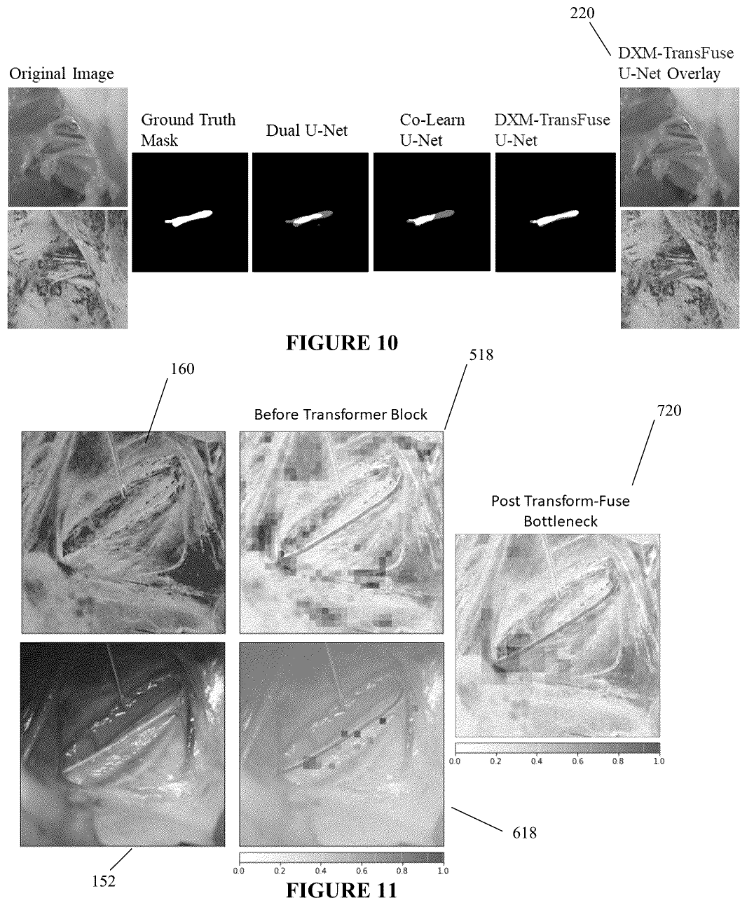

FIG. 8 shows predicted mask comparisons of single modality (Upper two rows: birefringence images, lower two rows: RGB images). FIG. 9 shows predicted mask comparisons between single modality and the proposed multi-modality networks. FIG. 10 shows predicted mask comparisons among different multi-modality networks. The Jet image is the birefringence map in Jet colorscale. Overlay the mask on images 150, 160 to provide the mask output 220. The image output 220a is output from the U-Net 212. Overlay image data 150, 160 on the image output 220a to obtain the DXM-Transfuse U-Net Overlay image data 220b.

Table 1 provides the comparison of the validation results between the network structures for each image representation. Regarding the birefringence images, the cross-attention U-Net had the highest Dice and F2 scores, while U-Net had better nerve detection metrics, including accuracy, sensitivity, and balanced accuracy. Regarding the RGB images, the cross-attention U-Net had higher accuracy, sensitivity, and F2 than the other models, the U-Net outperformed in specificity and balanced accuracy, and Attention U-Net showed the best in precision. When comparing between the image modalities, the models using birefringence images outperformed those with RGB images across all metrics. It is worth noting that due to resource limitations, only a single attention head was used in the cross-attention U-Net model.

TABLE 1
Validation result comparisons of different network structures for single modality.

| | | Detection metrics [Mean ± | | | | | | |
|---|---|---|---|---|---|---|---|---|
| | | Standard deviation %] | | | | | Segmentation | |
| Image | | | | | | Balanced | metrics | |
| representation | [symbol] | Accuracy | Sensitivity | Specificity | Precision | Accuracy | F2 | Dice |
| [symbol] | U-Net | [symbol] ± 7.69 | [symbol] ± 9.09 | 94.64 ± 6.59 | 98.31 ± 2.10 | [symbol] ± 6.36 | [symbol] ± 4.10 | [symbol] ± 4.14 |
| | Att. U-Net | 79.77 ± 7.50 | [symbol] ± 9.99 | [symbol] ± 6.59 | [symbol] ± 2.07 | 34.96 ± 6.04 | 72.17 ± 5.52 | [symbol] ± 4.95 |
| | Cross-Att. U-Net | [symbol] ± [symbol] | 77.46 ± [symbol] | 94.64 ± 6.59 | [symbol] ± 2.10 | [symbol] ± 6.53 | 72.79 ± 5.15 | [symbol] ± 4.47 |
| [symbol] | U-Net | [symbol] ± 6.54 | [symbol] ± 5.71 | [symbol] ± 11.09 | 95.02 ± [symbol] | [symbol] ± 8.17 | 63.19 ± 5.57 | 61.12 ± 5.40 |
| | Att. U-net | 72.35 ± [symbol] | 67.91 ± 3.50 | [symbol] ± [symbol] | 95.31 ± 2.69 | 77.92 ± 3.69 | 63.37 ± 3.02 | 60.94 ± 3.41 |
| | Cross-Att. U-net | [symbol] ± [symbol] | 71.27 ± 4.56 | [symbol] ± 12.11 | 93.79 ± 4.44 | 77.03 ± 6.59 | 63.65 ± 4.22 | 61.12 ± 4.35 |

[symbol]

indicates data missing or illegible when filed

FIGS. 8, 9 and 10 demonstrate the mask differences between ground truth and the predicted mask of its respective network. On the mask comparison images, the sections highlighted in blue are related to an over segmentation prediction, red where under segmentation occurred, and white where correctly segmented.

FIG. 8 illustrates the differences between the single-modality U-Net, the Attention U-Net, and the Cross-Attention U-Net approaches. The upper row demonstrates a challenging case with a birefringence image where it can be seen that the Attention U-Net was not able to detect the nerve, the U-Net had a lot of under-segmentation, but the CrossAttention U-Net was able to detect more segments of the nerve. The second row shows that in cases where all three models performed well with a birefringence image, the Cross-Attention U-Net still outperformed the others with minimal under-segmentation. The third row in FIG. 8 shows a challenging case with an RGB image in which the CrossAttention U-Net correctly detected a more significant part of the nerve with less over-segmentation than the U-Net, while all other architectures had some under-segmentation. The last row further demonstrates that the Cross-Attention U-Net minimized the under-segmentation error in cases where other models performed well with an RGB image. In general, introducing a cross-attention module is advantageous in extracting salient features and suppressing irrelevant information.

Table 2 provides the validation comparison between the dual modality networks. All three multi-modality networks had significantly outperformed single-modality networks across all metrics including detection and segmentation metrics (p<0.5). Furthermore, within the multi-modality networks, the proposed DXM-TransFuse U-net had the best detection and segmentation metrics.

TABLE 2
Validation result comparisons of different network structures for multi-modality.

| | | Detection metrics [Mean ± | | | | | | |
|---|---|---|---|---|---|---|---|---|
| | | Standard deviation %] | | | | | Segmentation | |
| Image | | | | | | Balanced | metrics | |
| representation | [symbol] | Accuracy | Sensitivity | Specificity | Precision | Accuracy | F2 | Dice |
| [symbol] + RGB | Duel U-Net | [symbol] ± 5.95 | [symbol] ± 7.27 | 94.64 ± 6.59 | [symbol] ± [symbol] | [symbol] ± 5.26 | 74.40 ± 4.46 | [symbol] ± 4.67 |
| | [symbol] U-Net | [symbol] ± 6.56 | [symbol] ± [symbol] | 95.50 ± 5.57 | 90.42 ± 1.96 | 89.30 ± 4.23 | [symbol] ± 3.63 | 71.13 ± 3.63 |
| | DXM-TransFuse | 88.34 ± 7.51 | [symbol] ± 9.21 | 97.50 ± [symbol] | 99.29 ± 1.43 | 91.72 ± 4.73 | 76.12 ± 3.40 | 72.10 ± 3.99 |
| | U-net* | | | | | | | |

(*is the proposed DXM-TransFuse U-net.) [symbol]
indicates data missing or illegible when filed

FIG. 9 shows that the proposed DXM-TransFuse U-net is able to leverage each individual modality's information to improve the overall nerve detection. As can be seen, all single modality networks, regardless of image representation, were unable to detect the nerve correctly. However, the DXM-TransFuse U-net was able to drastically improve the detection in such a case.

FIG. 10 illustrates the differences between the predicted masks of each multi-modal network. It can be seen that the proposed DXM-TransFuse U-net had shown higher performance in the segmentation task. Both the Dual U-Net and Co-Learn U-Net networks showed a lot of under segmentation, while the DXM-TransFuse U-net only slightly missed small portions of the nerve.

As shown in Table 1 and Table 2, our model has a significantly higher F2 score (p<0.5) when compared with other fusion and single modality methods, however, the Dice coefficient is not significant compared with Co-Learn U-Net. Nevertheless, the F2 score, which places more emphasis on under segmentation, would be a better predictor of the model performance with respect to nerve detection as it is more concerning if we incorrectly detected part of the nerve than overestimated the area of the nerve. Considering all these results, the proposed DXM-TransFuse U-net has the highest performance for multimodality, with the Cross-Attention U-Net also showing effectiveness for single-modality.

Table 3 shows inference times and the number of parameters of the single and multi-modality networks. Although multi-modality networks have an increase of at least 10 million parameters compared to single modality networks, the inference time of the DXM-Transfuse U-Net is almost ten milliseconds (ms) faster than that Cross-Att U-Net, which had the highest F2 score for single modality. With inference times of less than 30 ms for DXM-Transfuse U-Net, the network is processing at a little over 30 frames per second, which is sufficient for smooth visualization for nerve identification. In training, the parameters are being optimized, whereas in inference the optimal parameters are fixed and utilized.

### Network Specific Details.

TABLE 3

| CNN | Inference time (ms) | Number of parameters |
|---|---|---|
| U-Net | 17.76 ± 0.02 | 34,527,041 |
| Att. U-Net | 19.28 ± 0.04 | 34,878,573 |
| Cross-Att. U-Net | 49.18 ± 0.14 | 37,324,801 |
| Dual U-Net | 27.07 ± 0.04 | 47,068,289 |
| Co-Learn U-Net | 29.68 ± 0.01 | 56,506,497 |
| DXM-TransFuse U-Net | 29.30 ± 0.01 | 53,373,057 |

To further understand if the transformer block fusion can help with nerve segmentation, we looked at the Grad-Cam attributions at the layer before the transformer block and after the bottleneck (the point at which the transformer block and fusion of the features maps from each encoder have been completed). The case used in FIG. 10 had not performed well for the single modality network when using birefringence or RGB images (less than 47.5 F2 score) but had a 63.90 F2 score with DXM-Transfuse U-Net. In FIG. 11, we can see that before the Transformer block, neither encoder highlighted much of the area where the nerve was located. However, post bottleneck, more areas of the nerve were attributed, indicating that the transformation and fusion of the modalities helped focus on the area of interest. FIG. 11 shows layer Grad-Cam attributions for each encoder path before transformer block and post transform-fusion after bottleneck. Cyan color pixels represent ground truth mask of the nerve. As shown, merges to center line, and the (green) dots are features that are kept.

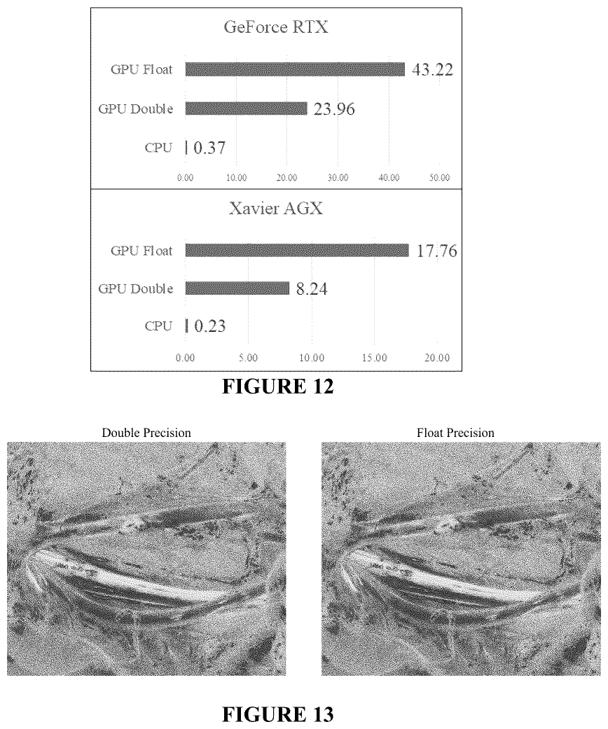

The execution times for the birefringence mapping calculations on the Windows machine are displayed in Table 4. The CPU implementation took on average 2739 milliseconds (ms) per image, while GPU with double precision took 41.74 ms and with float precision about 23.14 ms. The estimated frames per second (FPS) rate for each implementation can be viewed on the top half of FIG. 12 (estimated frames per second for different data types). The minimal gain by using the GPGPU implementation is 65.6×to as much as 118.4×.

TABLE 4
BIREFRINGENCE MAPPING EXECUTION TIMES.

| Device | Implementation | Execution Time (ms) |
|---|---|---|
| Windows Machine | CPU | 2739 |
| | GPU Double | 41.74 |
| | GPU Float | 23.14 |
| Xavier AGX | CPU | 4425 |
| | GPU Double | 121.4 |
| | GPU Float | 56.29 |

Large gains were also seen when executing the code on the Xavier AGX developer kit. As per Table 4, the CPU implementation took 4425 ms per image while GPU using double precision took 121.4 ms and when using float precision, it took 56.29 ms. On the Xavier device, the minimal gain was 36.4×, and the maximum gain of 78.6×. The estimated FPS rates are shown on the bottom half of FIG. 12. It should be noted that when using float precision, certain pixel outputs may not match that when using double precision. However, the percentage of mismatched pixels was less than 0.08% making it visually indistinguishable as shown in FIG. 13 (image outputs based on double and float precision).

TABLE 5
TOTAL EXECUTION TIMES WITH
DEEP LEARNING NETWORK.

| Device | Implementation | Execution Time (ms) |
|---|---|---|
| Windows Machine | CPU/F32 | 2771 |
| | GPU Double/F32 | 112.79 |
| | GPU Float/F32 | 96.02 |
| | GPU Float/F16 | 85.46 |

Table 5 provides the total execution times it took from initial raw data to mask predictions of the deep learning network. Implementation type is either CPU, GPU Double, or GPU Float for the birefringence mapping with the neural network using either a Float 32 or Float 16 data type precision.

On the Windows machine, the CPU implementation with the neural network using Float 32 (CPU/F32), the whole process could take up to 2771 ms. Moving to the GPU implementation drops to 112.79 ms, and using lower data type precisions, the framework can achieve 85.46 ms execution time.

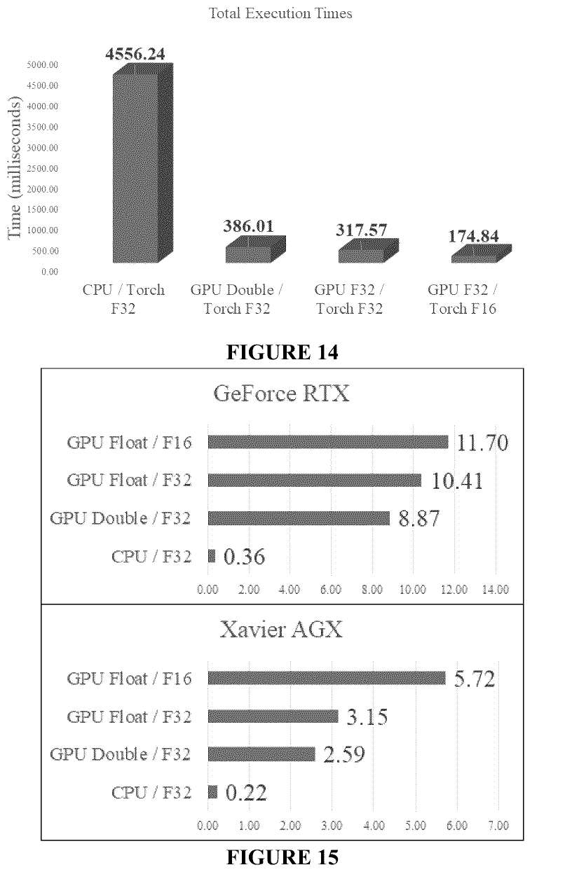

FIG. 14 is a comparison of total execution times with Deep Learning Networks on Xavier AGX Developer Kit, and FIG. 15 is an estimated FPS for complete flow of mask predictions. When running the aforementioned tests on the Xavier AGX, the CPU/F32 implementation took 4556 ms while the GPU Float/F16 implementation dropped to 174.84 ms, as depicted on FIG. 14. The improvement on the Windows machine was upwards of 32.4×achieving 11+FPS and on Xavier AGX 26.4×with close to 6 FPS as shown on FIG. 15. Using a lower data type precision for the deep learning inferences did not have an effect on the final predicted masks.

### Materials

Animal procedures and dataset preparation. Sample nerve images were acquired at the Children's National Research Animal Facility under the approval of the Institutional Animal. Care and Use Committee (IACUC #30591). After euthanasia of the living animals (N=4), the cervical incisions on the central neck of each pig were performed using standard surgical instruments: sharp dissection by scalpel, blunt dissection by scissors/forceps, and coagulation by electrocoagulation. The ventral portion of the superficial neck muscle was exposed. Vagus nerves, recurrent laryngeal nerves, and superior laryngeal nerves were dissected and targeted for imaging using an in-house dual-RGB/polarimetric imaging system (Ning et al., 2021) similar to the previous work (Cha et al., 2018).

### CONCLUSIONS

In this study, we proposed the usage of birefringence images with deep learning inference to help with the detection of nerves. Additionally, we proposed the DXM-TransFuse U-net that could fuse information from multi-modal medical images. The study findings showed that leveraging birefringence images outperforms its RGB counterpart for nerve detection and segmentation. Additionally, introducing the cross-attention module on a single modality network improved the detection and segmentation of nerve structures on the birefringence images. Furthermore, by employing parallel encoders to extract the feature maps of each modality independently and applying the Transformer module to fuse the information of each, the network with Transformer was able to improve its segmentation of nerve structures further, resulting in better overall detection.

However, given the relatively small sample size in this study due to the difficulty of acquiring a large dataset of clinical nerve images, challenges were present in developing and validating our models. To ensure the robustness of the models, we applied the 5-fold validation during the experiments. Furthermore, although data augmentation was applied in this study, the model's generalization can be enhanced further by increasing the dataset in future studies. Nonetheless, our research will be constructive in combining different image modalities for nerve image segmentation.

Further modifications to the network can be implemented by extending the usage of the Transformer module at each skip connection layer to strengthen the cross-modal interactions between the modalities. Due to the differences in the variance in brightness and contrast of each imaging modality, further investigations to determine optimal color augmentation criteria for each modality are recommended for future studies.

This disclosure also demonstrates the benefits of translating a birefringence mapping from a CPU to GPGPU implementation that achieves a processing time boost of 118+times and provides a smoother visualization experience that can benefit with identifying nerves. Combined with our prior work with deep learning networks, DXM-TransFuse U-net [15], this framework can enable real-time nerve tissue identification and visualization during open surgeries with the recent advancement with polarimetric imaging systems [2]. Furthermore, this technology also provides a bright possibility in advancing endoscopic imaging as well as telesurgical applications, where real-time image analysis and visualization will significantly improve surgical outcomes.

Though using a float precision data type for birefringence mapping may result in a minor difference from that of double-precision, we believe the 2×gains outweigh the pixel difference, as higher FPS rates would result in a better end user experience. Further optimizations can be performed by identifying the ideal CUDA grid and block dimensions for the device where the code will be executed. Additionally, the following points should be considered when including a neural network in the overall flow: 1) minimize the amount of pre-processing required, and 2) avoid transfers between the host and device. Therefore, it is recommended that future investigations are performed using the birefringence output as a direct input to the neural network, avoiding any pre-processing activities and potentially intermediate data transfers.

The system and method of the present disclosure (e.g., the GPGPU and/or the GPU) can be implemented by a processing device to perform various functions and operations in accordance with the disclosure. The processing device can be, for example, a computer, computing device, processor, personal computer (PC), server or mainframe computer. In addition to the processing device, computer hardware may include one or more of a wide variety of components or subsystems including, for example, a co-processor, input devices (keyboard, touchscreen, mouse), monitors, wired or wireless communication links, and a memory or storage device such as a database. The system can be a network configuration or a variety of data communication network environments using software, hardware or a combination of hardware and software to provide the processing functions. Unless indicated otherwise, the process is preferably implemented automatically by the processor substantially in real time without delay or manual action.

All or parts of the system and processes can be implemented at the processing device by software or other machine executable instructions which is stored on or read from computer-readable media for performing the processes described above. Computer readable media may include, for instance, one or more: hard disks, floppy disks, and CD-ROM; a carrier wave received from the Internet; or other forms of computer-readable memory such as read-only memory (ROM) or random-access memory (RAM), solid-state, analog or other memories; optical and/or magnetic media; a centralized or distributed database; and/or caches.

The processes can be implemented in a variety of ways including modules, programs, applications, scripts, processes, threads or code sections that interrelate with each other. The program modules can be commercially available software, discrete electrical components or customized hardwired application specific integrated circuits (ASIC).

In one embodiment, a Zeiss OPMI MDO S5 surgical microscope (surgicalmicroscopes.com) can be used as the camera system. And, a Jetson Xavier (nvidia.com) can be used for real-time image segmentation.

The foregoing description and drawings should be considered as illustrative only of the principles of the disclosure, which may be configured in a variety of shapes and sizes and is not intended to be limited by the embodiment herein described. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.
References are incorporated herein by reference. [1] G. Antoniadis, T. Kretschmer, M. T. Pedro, R. W. König, C. P. Heinen, and H.-P. Richter, “Iatrogenic nerve injuries: prevalence, diagnosis and treatment,” Deutsches Arzteblatt International, vol. 111, no. 16, p. 273, 2014. [2] J. Cha, A. Broch, S. Mudge, K. Kim, J.-M. Namgoong, E. Oh, and P. Kim, “Real-time, label-free, intraoperative visualization of peripheral nerves and micro-vasculatures using multimodal optical imaging techniques,” Biomedical Optics Express, vol. 9, no. 3, pp. 1097-1110, 2018. [3] A. Mishra, A. Agarwal, G. Agarwal, and S. Mishra, “Total thyroidectomy for benign thyroid disorders in an endemic region,” World journal of surgery, vol. 25, no. 3, pp. 307-310, 2001. [4] B. Bai and W. Chen, “Protective effects of intraoperative nerve monitoring (ionm) for recurrent laryngeal nerve injury in thyroidectomy: meta-analysis,” Scientific reports, vol. 8, no. 1, pp. 1-11, 2018. [5] J. M. Culp and G. Patel, “Recurrent laryngeal nerve injury,” StatPearls [Internet], 2021. [6] S.-M. Kim, S. H. Kim, D.-W. Seo, and K.-W. Lee, “Intraoperative neurophysiologic monitoring: basic principles and recent update,” Journal of Korean medical science, vol. 28, no. 9, pp. 1261-1269, 2013.

[7] M. Biscevic, A. Sehic, and F. Krupic, “Intraoperative neuromonitoring in spine deformity surgery: modalities, advantages, limitations, medicolegal issues-surgeons' views,” EFORT Open Reviews, vol. 5, no. 1, pp. 9-16, 2020. [8] M. A. Whitney, J. L. Crisp, L. T. Nguyen, B. Friedman, L. A. Gross, P. Steinbach, R. Y. Tsien, and Q. T. Nguyen, “Fluorescent peptides highlight peripheral nerves during surgery in mice,” Nature biotechnology, vol. 29, no. 4, pp. 352-356, 2011. [9] S. Wachsmann-Hogiu, T. Weeks, and T. Huser, “Chemical analysis in vivo and in vitro by raman spectroscopy—from single cells to humans,” Current opinion in biotechnology, vol. 20, no. 1, pp. 63-73, 2009. G. C. Langhout, K. F. Kuhlmann, M. W. Wouters, J. A. van der Hage, F. van Coevorden, M. Mu “ller, T. M. Bydlon, H. J. Sterenborg, B. H. Hendriks, and T. J. Ruers, “Nerve detection during surgery: optical spectroscopy for peripheral nerve localization,” Lasers in medical science, vol. 33, no. 3, pp. 619-625, 2018. S. Vinayahalingam, T. Xi, S. Berge', T. Maal, and G. de Jong, “Automated detection of third molars and mandibular nerve by deep learning,” Scientific reports, vol. 9, no. 1, pp. 1-7, 2019. O. Ronneberger, P. Fischer, and T. Brox, “U-net: Convolutional networks for biomedical image segmentation,” in International Conference on Medical image computing and computer-assisted intervention. Springer, 2015, pp. 234-241.

[13]M. Barberio, T. Collins, V. Bencteux, R. Nkusi, E. Felli, M. G. Viola, J. Marescaux, A. Hostettler, and M. Diana, “Deep learning analysis of in vivo hyperspectral images for automated intraoperative nerve detection,” Diagnostics, vol. 11, no. 8, p. 1508, 2021. B. M. Henry, M. J. Graves, J. Vikse, B. Sanna, P. A. Pekala, J. A. Walocha, M. Barczyn'ski, and K. A. Tomaszewski, “The current state of intermittent intraoperative neural monitoring for prevention of recurrent laryngeal nerve injury during thyroidectomy: a prismacompliant systematic review of overlapping meta-analyses,” Langenbeck's archives of surgery, vol. 402, no. 4, pp. 663-673, 2017. B. Xie, G. Milam, B. Ning, J. Cha, and C. H. Park, “Dxm-transfuse u-net: Dual cross-modal transformer fusion u-net for automated nerve identification,” Computerized Medical Imaging and Graphics, p. 102090, 2022. J. Qi, M. Ye, M. Singh, N. T. Clancy, and D. S. Elson, “Narrow band 3×3 mueller polarimetric endoscopy,” Biomedical optics express, vol. 4, no. 11, pp. 2433-2449, 2013.

Cha, J., Broch, A., Mudge, S., Kim, K., Namgoong, J.-M., Oh, E., Kim, P., 2018. Real-time, label-free, intraoperative visualization of peripheral nerves and micro-vasculatures using multimodal optical imaging techniques. Biomed. Opt. Express 9, 1097-1110. Dolz, J., Desrosiers, C., and Ayed, I. B., 2018b. Ivd-net: Intervertebral disc localization and segmentation in mri with a multi-modal unet. In: Proceedings of the International Workshop and Challenge on Computational Methods and Clinical Applications for Spine Imaging. pp. 130-143. Springer. Dolz, J., Ayed, I. B., and Desrosiers, C., 2018a. Dense multi-path u-net for ischemic stroke lesion segmentation in multiple image modalities. In: Proceedings of the International MICCAI Brainlesion Workshop 271-282. Springer. Henry, B. M., Graves, M. J., Vikse, J., Sanna, B., Pkala, P. A., Walocha, J. A., Barczyn'ski, M., Tomaszewski, K. A., 2017. The current state of intermittent intraoperative neural monitoring for prevention of recurrent laryngeal nerve injury during thyroidectomy: a prisma-compliant systematic review of overlapping meta-analyses. Langenbeck's Arch. Surg. 402, 663-673. Kretschmer, T., Antoniadis, G., Braun, V., Rath, S. A., Richter, H.-P., 2001. Evaluation of iatrogenic lesions in 722 surgically treated cases of peripheral nerve trauma. J. Neurosurg. 94, 905-912.

Kumar, A., Fulham, M., Feng, D., Kim, J., 2020. Co-learning feature fusion maps from pet-ct images of lung cancer. IEEE Trans. Med. Imaging 39, 204-217. https://doi. org/10.1109/TMI.2019.2923601. Ning, B., Kim, W. W., Katz, I., Park, C. H., Sandler, A. D., Cha, J., 2021. Improved nerve visualization in head and neck surgery using mueller polarimetric imaging: preclinical feasibility study in a swine model. Lasers Surg. Med. 53 (10), 1427-1434. Oktay, O., Schlemper, J., Folgoc, L. L., Lee, M., Heinrich, M., Misawa, K., Mori, K., McDonagh, S., Hammerla, N. Y., Kainz, B. et al., 2018. Attention u-net: Learning where to look for the pancreas. arXiv preprint arXiv: 1804.03999. Petit, O., Thome, N., Rambour, C., and Soler, L., 2021. U-net transformer: Self and cross attention for medical image segmentation. arXiv preprint arXiv: 3.06104. Ronneberger, O., Fischer, P., and Brox, T., 2015. U-net: Convolutional networks for biomedical image segmentation. In: Proceedings of the International Conference on Medical Image Computing and Computer-assisted intervention. pp. 234-241. Springer.

Tsai, Y.-H. H., Bai, S., Liang, P. P., Kolter, J. Z., Morency, L.-P., and Salakhutdinov, R., 2019. Multimodal transformer for unaligned multimodal language sequences. In: Proceedings of the Conference on Association for Computational Linguistics. Meeting 6558. NIH Public Access volume 2019. Vaswani, A., Shazeer, N., Parmar, N., Uszkoreit, J., Jones, L., Gomez, A. N., Kaiser, L., and Polosukhin, I. (2017). Attention is all you need. In: Proceedings of the Advances in Neural Information Processing Systems. pp. 5998-6008. Zhou, Z., Siddiquee, M. M. R., ++Tajbakhsh, N., and Liang, J., 2018. Unet: a nested u-net architecture for medical image segmentation. In: Proceedings of the Deep Learning in Medical Image Analysis and Multimodal Learning for Clinical Decision Support. pp. 3-11. Springer.

1. A nerve detection system comprising:
a general-purpose graphics processing unit (GPGPU) configured to receive raw polarized data and raw RGB image data, generate a birefringence output based on the raw polarized data, and simultaneously process the raw RGB data and the birefringence output to provide nerve detection information.

2. The system of claim 1, further comprising a transfuse network configured to simultaneously process the raw RGB data and the birefringence output to provide the nerve detection information

3. The system of claim 1, further comprising an image acquisition device which comprises a dual RGB and polarimetric imaging device, said GPGPU receiving the raw polarized data and the raw RGB data from said image acquisition device.

4. The system of claim 1, said GPGPU generating a birefringence map based on the raw polarized data.

5. The system of claim 3, said GPGPU comprising a deep learning network trained on domain-specific data to optimize the deep learning network to produce a fast inference network output.

6. The system of claim 3, said GPGPU providing a nerve segmentation mask.

7. The system of claim 3, wherein the raw polarized data has a plurality of output pixels, and said GPGPU performs birefringence calculations in parallel for each output pixel of the plurality of output pixels, to obtain a birefringence output.

8. The system of claim 7, wherein said GPGPU uses a BRF representation of the birefringence output and the RGB image data to identify nerve structure.

9. The system of claim 8, wherein the GPGPU performs birefringence mapping on the raw polarized data, normalizes the birefringence mapping and applies a BRF colormap to obtain the BRF representation.

10. The system of claim 1, said GPGPU having a transformer block that fuses a birefringence modality of the birefringence output and an RGB modality of the RGB image.

11. The system of claim 10, said GPGPU providing a birefringence map based on the fused birefringence modality and RGB modality.

12. A method for nerve detection comprising:
receiving at a general-purpose graphics processing unit (GPGPU), raw polarized data and raw RGB image data;
generating at the GPGPU, a birefringence output based on the raw polarized data; and
simultaneously processing the raw RGB data and the birefringence output to provide nerve detection information.

13. The method of claim 12, further comprising simultaneously processing, at a transfuse network configured, the raw RGB data and the birefringence output and providing the nerve detection information

14. The method of claim 12, further comprising capturing the raw polarized data and raw RGB image data from a dual RGB and polarimetric imaging device.

15. The method of claim 12, further comprising generating at the GPGPU, a birefringence map based on the raw polarized data.

16. The method of claim 14, the GPGPU comprising a deep learning network that can be trained on domain-specific data to optimize the network for the data set a produce a fast inference network output.

17. The method of claim 14, providing a nerve segmentation mask at the GPGPU.

18. The method of claim 14, wherein the raw polarized data has a plurality of output pixels, and the GPGPU performs birefringence calculations in parallel for each output pixel of the plurality of output pixels, to obtain a birefringence output.

19. The method of claim 12, wherein the GPGPU uses a BRF representation of the birefringence output and the RGB image data to identify nerve structure.

20. The method of claim 19, wherein the GPGPU performs birefringence mapping on the raw polarized data, normalizes the birefringence mapping and applies a BRF colormap to obtain the BRF representation.

21. The method of claim 12, the GPGPU having a transformer block, and fusing at the transformer block, a birefringence modality of the birefringence output and an RGB modality of the RGB image.

22. The method of claim 21, the GPGPU providing a birefringence map based on the fused birefringence modality and RGB modality.
